(19) United States
(12) Patent Application Publication
(10) Pub. No.: US 2026/0363711 A1
(43) Pub. Date: Nov. 19, 2026

(54) GRAFT CUTTING DEVICE AND METHODS OF USING THE SAME

(71) Applicants: THE TEXAS A&M UNIVERSITY SYSTEM, College Station, TX (US); THE METHODIST HOSPITAL, Houston, TX (US)

(72) Inventors: Robert Kenneth Sims, IV, Houston, TX (US); Eugene L. Alford, Houston, TX (US)

(21) Appl. No.: 19/143,992

(22) PCT Filed: Dec. 28, 2023
(86) PCT No.: PCT/US23/86191

§ 371 Date: Jun. 27, 2025

Related U.S. Application Data
(60) Provisional application No. 63/477,426, filed on Dec. 28, 2022.

(51) Int. Cl. A61B 17/16; A61B 17/00; A61B 17/17

(52) U.S. Cl. CPC A61B 17/1635; A61B 17/17; A61B 2017/00858

(57) ABSTRACT

A graft cutting device includes a base, a cutting arm, and a plurality of fixators. The base has a cutting board section that defines a plurality of receptacles and pair of legs that each define a slot. The cutting arm is disposed between the pair of legs and is movably coupled to the base. The cutting arm includes a first arm member, a second arm member, a first blade, a second blade, and a spacer. The first blade and the second blade are disposed between the first arm member and the second arm member, and the spacer is disposed between the first blade and the second blade. The fixators are received within a respective receptacle and are configured to fix a piece of donor material to the cutting board section such that the dimensions of the piece of donor material are retained while fixed to the cutting board section.

### CROSS-REFERENCE TO RELATED APPLICATIONS

This application claims priority to and the benefit of U.S. Provisional Patent Application Ser. No. 63/477,426, filed Dec. 28, 2022. The entire contents of the above application are hereby incorporated by reference.

### BACKGROUND

### 1. Technical Field

The present disclosure relates to graft cutting devices and, more specifically, graft cutting devices for cartilage.

### 2. Discussion of Related Art

Cartilage harvesting and carving is a common part of facial plastic and reconstructive surgery-especially rhinoplasty. Typically, this involves using a scalpel to cut a particular shape from either a frozen piece of cadaveric cartilage, irradiated cartilage, or autologous cartilage. Often, the cartilage is derived from donor rib, commonly known as an autologous costal cartilage graft. Rhinoplasty is very architectural, meaning the form and function of the nose depends on its “hidden” bone and cartilage structural support system, with the nasal septum being the center-piece support feature. Sometimes, patients do not have enough natural support features available to allow for the desired outcome, especially in revision rhinoplasty. Costal cartilage grafting may be used to form support features to create the desired function and appearance.

Costal cartilage grafting involves using thin slices of costal cartilage to augment the nasal support system in many ways. For example, columellar strut grafts may be used for better nasal tip support, or caudal-septal grafts or batten grafts may be used to prevent internal and/or external nasal valve collapse. It is important that the carved cartilage is the right size and shape. Otherwise, the resulting form and function of the nose may be worse than intended. Carving rib cartilage is a skill and presents challenges for the most seasoned facial plastic surgeons because cartilage is prone to warping. Sometimes, the cartilage can warp after it is grafted into the patient's nose, leaving a less than desirable outcome. The most common complication of costal cartilage grafts is warping at a rate of 10%.

Intrinsic properties of cartilage contribute to warping. Nasal cartilage is a hyaline cartilage making it great for support because it is not too rigid nor too elastic. The cells are called chondrocytes, and they are heterogeneously scattered throughout the tissue. Collagen and other extra-cellular matrix proteins fill in the inter-chondrocyte space. The cartilage has a high-water composition, but the water density is not evenly distributed through the tissue, and the mechanical properties are not uniform. On the other hand, costal cartilage has dynamic, heterogenous mechanical properties with the “short” side experiencing intrinsic compressive load and the “long” side experiencing tensile force. These properties give costal cartilage “memory,” meaning the cartilage tends to return to its natural shape even after manipulation. This makes cartilage carving somewhat of an unpredictable process, and lack of precise carving and inaccurate anticipation of tissue behavior can lead to adverse functional and cosmetic outcomes, such as a curved nose appearance.

### SUMMARY

Applicants have recognized a need for precise cutting of graft cartilage for predictability of surgical outcomes. In an aspect of the present disclosure, a graft cutting device includes a base, a cutting arm, and a plurality of fixators. The base has a cutting board section and pair of legs. The cutting board section defines a plurality of receptacles and the pair of legs each define a slot. The cutting arm is disposed between the pair of legs and is movably coupled to the base within the slots such that the cutting arm rotates or translates with respect to the base between an open position in which the cutting arm is disengaged from the cutting board section and a closed position in which the cutting arm is engaged with the cutting board section. The cutting arm includes a first arm member, a second arm member, a first blade, a second blade, and a spacer. The first blade and the second blade are disposed between the first arm member and the second arm member. The spacer is disposed between the first blade and the second blade. The first blade and the second blade are spaced apart by the spacer. Each fixator of the plurality of fixators are received within a respective receptacle of the plurality of receptacles. The plurality of fixators are configured to fix a piece of donor material to the cutting board section such that the dimensions of the piece of donor material are retained while fixed to the cutting board section.

In aspects, each fixator of the plurality of fixators include a first holding member, a second holding member, and a spring extending between the first holding member and the second holding member. The spring may urge the first holding member and the second holding member apart such that when the fixator is received within a respective receptacle of the cutting board section the fixator is adjustably secured to the base within the receptacle by the first holding member and the second holding member urged against a pair of side walls of the receptacle by the spring. The first holding member and the second holding member may each include a plurality of teeth projecting therefrom. Each tooth of the plurality of teeth may have a conical profile.

In some aspects, the graft cutting device includes a cutting bed disposed within a bed chamber defined between a top surface of the cutting board section and a bed platform of the cutting board section. A top surface of the cutting bed may be coplanar with the top surface of the cutting board section. The top surface of the cutting bed may be recessed below the top surface of the cutting board section or raised above the top surface of the cutting board section.

In certain aspects, the first arm member, the second arm member, and the spacer each define a cut-out top expose a portion of the first blade and the second blade to form a cutting zone of the cutting arm. The first blade, the second blade, and the spacer may define a graft pocket therebetween. The graft pocket has a width that may define a thickness of a piece of graft material cut from the piece of donor material. The width of the graft pocket may be defined by a thickness of the spacer.

In particular aspects, the first blade and the second blade have a single beveled edge. The first blade and the second blade may have a double beveled edge. The spacer may have a thickness in the range of 0.5 millimeters to 3 millimeters. The graft cutting device may include another spacer disposed between the first blade and the second blade to increase or decrease a cut thickness of the cutting arm.

In aspects, a graft cutting kit includes a graft cutting device and a plurality of spacers. Each of the spacers may be disposable between the first blade and the second blade to increase to increase or decrease a cut thickness of the graft cutting device.

In another aspect of the present disclosure, a graft cutting device includes a base, a cutting arm, and a plurality of fixators. The base has a cutting board section. The cutting arm is movably coupled to the base. The cutting arm is configured to cut a piece of graft material from a piece of donor material. The plurality of fixators are adjustably secured to the base. The fixators are configured to fix the piece of donor material to the cutting board section such that the piece of donor material is undeformed by the fixators while fixed to cutting board section.

In aspects, the cutting arm includes a spacer configured to define a thickness of the piece of graft material cut from the piece of donor material. The base may define a plurality of receptacle to receive a fixator of the plurality of fixators. Each receptacle may have a length. The fixator may be secured to the base within the receptacle such that the position of the fixator relative the base is adjustable along the length of the fixator. Each fixator may include a spring configured to urge a portion of the fixator against a pair of side wall of a receptacle such that the fixator is adjustably secured to the base.

In some aspects, the base includes a cutting bed inserted into the cutting board section of the base. The cutting bed may have nubs configured to the raise the piece of donor material such that there is clearance between the cutting bed and the piece of donor material.

In certain aspects, the base of graft cutting device may include a pair of legs extending outwardly therefrom. Each leg of the pair of legs may define a slot. The cutting arm may be movably coupled to the pair of legs within each slot such that the cutting arm is capable of rotating or translating with respect to the base. The base may include a plurality of clamp points configured to allow for securement of the base to a work surface. The base may include a cut guide configured to align a blade of the cutting arm relative to the piece of donor material such that the piece of graft material cut therefrom has uniform thickness.

In another aspect of the present disclosure, a fixator for a graft cutting device includes a first holding member, a second holding member, and a spring. The spring extends between the first holding member and the second holding member. The spring urges the first holding member and the second holding member away from each other. The fixator is configured to fix a piece of donor material the graft cutting device such that the piece of donor material is undeformed by the fixator.

In aspects, the first holding member and the second holding member are made from a high friction material. The high friction material may be a silicone-based material.

In some aspects, the first holding member and the second holding member each include a plurality of teeth projecting therefrom. Each tooth of the plurality of teeth of the first holding member and the second holding member may have a conical profile.

In another aspect of the present disclosure, a method of using a graft cutting device includes fixing a piece of donor material to a cutting board section of a base of the graft cutting device such that the dimensions of the piece of material are retained and cutting a piece of graft material from the piece of donor material by moving a cutting arm of the graft cutting device from an open position thereof to a closed position thereof.

In aspects, fixating the piece of donor material comprises adjusting a plurality of fixators of the graft cutting device to fix the piece of donor material. Adjusting the plurality of fixators may include positioning each fixator within a respective receptacle of a plurality of receptacles defined by the base.

In some aspects, the method includes increasing or decreasing a cut thickness of the graft cutting device. Increasing or decreasing the cut thickness of the graft cutting device may include changing a first spacer of the cutting arm to a second spacer. The first spacer may have a thickness different from the second spacer. Increasing or decreasing the cut thickness of the graft cutting device may include adding or removing a spacer of the cutting arm. The cutting arm may include a plurality of spacers. Each spacer of the plurality of spacers may have the same thickness.

In another aspect of the present disclosure, a graft cutting device may include a base, a cutting arm, and a clamp. The base has a cutting board section and pair of legs. The pair of legs each define a slot. The cutting arm is disposed between the pair of legs and movably coupled to the base within the slots such that the cutting arm rotates or translates with respect to the base between an open position in which the cutting arm is disengaged from the cutting board section and a closed position in which the cutting arm is engaged with the cutting board section. The cutting arm has a first arm member, a second arm member, a blade, a spacer, and a take up washer. The blade is disposed between the first arm member and the second arm member. The spacer is disposed adjacent to the first arm member or the second arm member. The take-up washer is disposed between one of the legs of the pair of legs and one of the spacer, the first arm member, or the second arm member. The take-up washer is configured to maintain alignment of the cutting arm relative to a piece of donor material to cut a uniform piece of graft material therefrom. The clamp is configured to fix the piece of donor material to the cutting board section such that the dimensions of the piece of donor material are retained while fixed to the cutting board section.

In aspects, the base has a pair of guide rail each defining a guide channel. Each guide rail may be opposite a respective leg of the pair of legs. Each guide channel may receive a knob of one of the first arm member or the second arm member. When the spacer is disposed adjacent to the first arm member the cutting arm may be configured to cut the piece of graft material to have a first thickness and when the spacer is disposed adjacent to the second arm member the cutting arm may be configured to cut the piece of graft material to have a second thickness different from the first thickness.

In certain aspects, the clamp includes a stopper wall, a clamp wall, and a clamp screw. The stopper wall may be disposed on the cutting board section of the base. The clamp wall may be disposed on the cutting board section of the base opposite the stopper wall. The clamp wall and the stopper wall may define a clamp chamber therebetween. The clamp screw may be adjustably coupled to the clamp wall such that the clamp screw is configured to engage the piece of donor material disposed within the clamp chamber and urge the piece of donor material against the stopper wall.

Further, to the extent consistent, any of the embodiments or aspects described herein may be used in conjunction with any or all of the other embodiments or aspects described herein.

### BRIEF DESCRIPTION OF THE DRAWINGS

Various aspects of the present disclosure are described hereinbelow with reference to the drawings, which are not necessarily drawn to scale, which are incorporated in and constitute a part of this specification, wherein:

FIG. 1 is a side perspective of a graft cutting device in accordance with embodiments of the present disclosure with a cutting arm a closed position thereof;

FIG. 2 is a side perspective view of the graft cutting device of FIG. 1 with the cutting arm an open position thereof;

FIG. 3 is the top view of the graft cutting device of FIG. 1 having a base with a different cutting board section and the cutting arm between the open position and the closed position;

FIG. 4 is a rear view of the graft cutting device of FIG. 3;

FIG. 5 is a perspective view of the base of FIG. 3;

FIG. 6 is a side view of the base of FIG. 5;

FIG. 7 is a detail view of the cutting arm of FIG. 1;

FIG. 8 is a perspective view of a spacer of the cutting arm of FIG. 7;

FIGS. 9-12 are side views and cross-sectional views of rectangular blades in accordance with embodiments of the present disclosure;

FIGS. 13-5 are side views of circular blades in accordance with embodiments of the present disclosure;

FIGS. 16 and 17 are side views of hemi-spherical blades in accordance with embodiments of the present disclosure;

FIG. 18 is a front view of a fixator in accordance with embodiments of the present embodiments;

FIG. 19 is a detail view of a fixator of FIG. 2;

FIG. 20 is a perspective view of another graft cutting device in accordance with embodiments of the present disclosure;

FIG. 21 is a perspective view of another base in accordance with embodiments of the present disclosure;

FIG. 22 is a perspective view of another graft cutting device in accordance with embodiments of the present disclosure;

FIG. 23 is an exploded view of the graft cutting device of FIG. 22;

FIG. 24 is a detail view of a clamp of the graft cutting device of FIG. 22; and

FIG. 25 is a flow chart illustrating a method of using a graft cutting device in accordance with embodiments of the present disclosure.

### DETAILED DESCRIPTION

The present disclosure will now be described more fully hereinafter with reference to example embodiments thereof with reference to the drawings in which like reference numerals designate identical or corresponding elements in each of the several views. These example embodiments are described so that this disclosure will be thorough and complete, and will fully convey the scope of the disclosure to those skilled in the art. Features from one embodiment or aspect can be combined with features from any other embodiment or aspect in any appropriate combination. For example, any individual or collective features of method aspects or embodiments can be applied to apparatus, product, or component aspects or embodiments and vice versa. The disclosure may be embodied in many different forms and should not be construed as limited to the embodiments set forth herein; rather, these embodiments are provided so that this disclosure will satisfy applicable legal requirements. As used in the specification and the appended claims, the singular forms “a,” “an,” “the,” and the like include plural referents unless the context clearly dictates otherwise. In addition, while reference may be made herein to quantitative measures, values, geometric relationships or the like, unless otherwise stated, any one or more if not all of these may be absolute or approximate to account for acceptable variations that may occur, such as those due to manufacturing or engineering tolerances or the like.

As used herein, the term “clinician” refers to a doctor, a nurse, or any other care provider and may include support personnel. “Donor material” refers to a piece of material that a material to be grafted on to a patient is cut from or originates. “Graft material” refers to the material to be grated on to a patient.

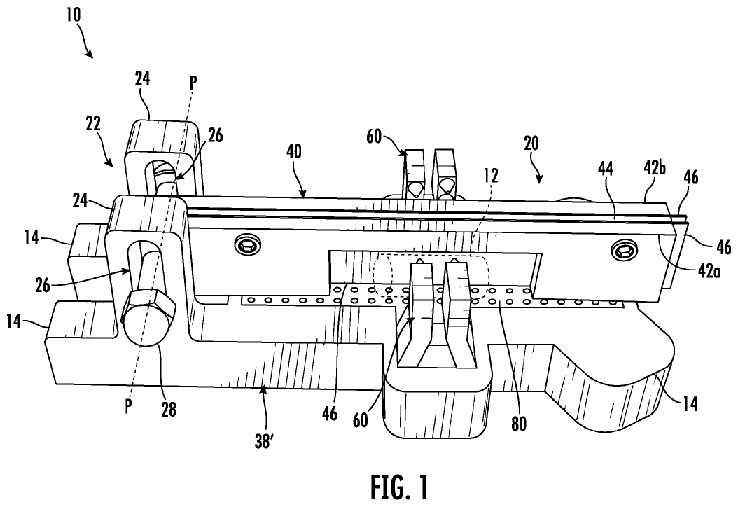

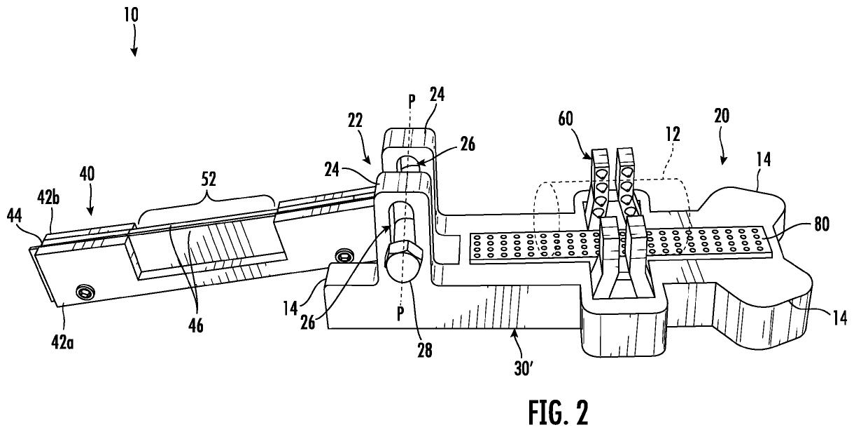

Referring now to FIGS. 1 and 2, a graft cutting device 10 in accordance with embodiments of the present disclosure is shown. The graft cutting device 10 includes a base 20, a cutting arm 40, fixators 60, and a cutting bed 80. A piece of donor material 12, e.g., cartilage, may be positioned on the base 20 and fixed in place by the fixators 60 to the cutting bed 80. The cutting arm 40 may be moved from an open position (FIG. 2) to a closed position (FIG. 1) to cut a piece of graft material from the donor material 12 for use in surgery, e.g., facial plastic or reconstructive surgery. In the open position the cutting arm 40 is disengaged from a cutting board section 30 of the base 20 to allow fixation of the donor material 12 to the cutting bed 80 and in the closed position the cutting arm 40 is engaged with the cutting board section 30 such that blades 46 of the cutting arm 40 may cut entirely through the donor material 12.

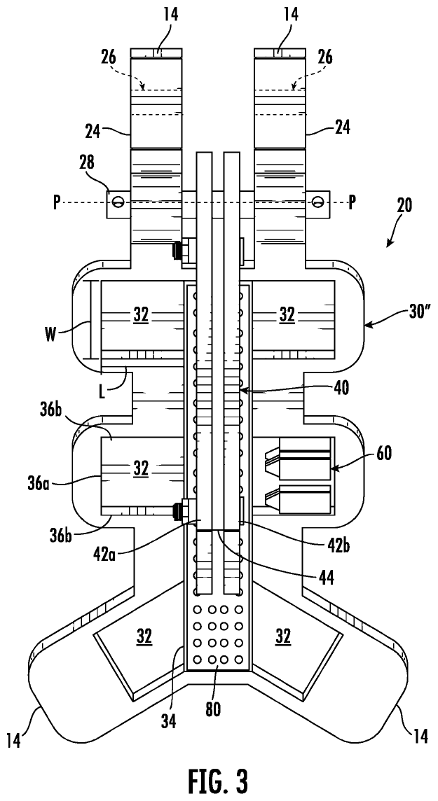

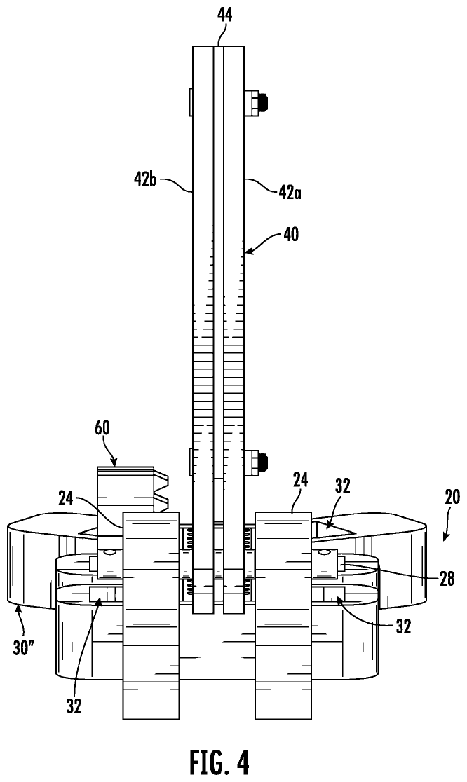

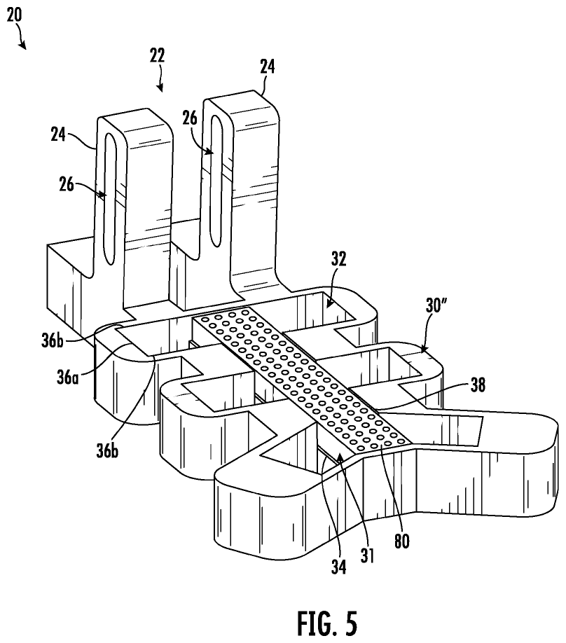

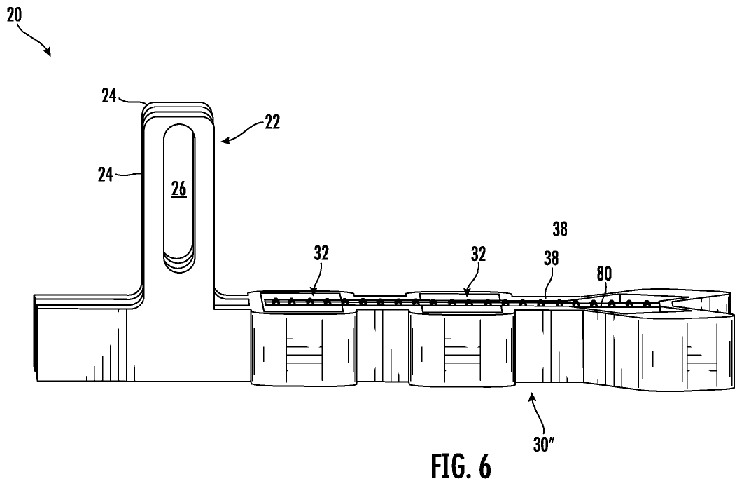

With additional reference to FIGS. 3-6, the base 20 includes an arm mount 22 the cutting board section 30. The arm mount 22 includes a pair of legs 24 spaced apart from each other to receive the cutting arm 40 and facilitate movement of the cutting arm 40 between the closed position and the open position. The legs 24 may extend from the base 20 vertically or horizontally. Each leg of the pair of legs 24 define a slot 26 to receive a pin 28 to movably couple the cutting arm 40 to the base 20. The slots 26 provide for translation or rotation of the cutting arm 40 with respect to the base 20. In some embodiments, the base 20 includes clamp points 14 to secure the graft cutting device 10 to a work surface, e.g., a table or workbench, to further stabilize the graft cutting device 10. The base 20 may be made of a sterilizable material to allow for repeated use. For example, the base 20 may be made of medical grade steel, titanium, aluminum, metal alloys, ceramics, or plastics. In certain embodiments, the base 20 may include a lock mechanism (not shown) configured to lock the cutting arm 40 in the open position or the closed position.

The cutting board section 30 supports a piece of donor material 12 during cutting of a piece of graft material. The cutting board section 30 defines a plurality of receptacles 32 to receive the fixators 60 and includes a cutting platform 34 to support the cutting bed 80. The cutting board section 30 may be a cutting board section 30′ including two receptacles 32, as shown in FIGS. 1 and 2, or may be a cutting board section 30″ including six receptacles 32, as shown in FIGS. 3-6. In certain embodiments, the cutting board section 30 may include more than six receptacles 32. The receptacles 32 are defined between interior walls 36 and the cutting platform 34. The receptacles 32 have a length L defined from a rear wall 36a to the cutting platform 34 and a width W defined between a pair of side walls 36b. The cutting platform 34 extends centrally along the cutting board section 30. The cutting platform 34 is recessed from a top surface 38 of the cutting board section 30 to define a bed chamber 31 to receive the cutting bed 80.

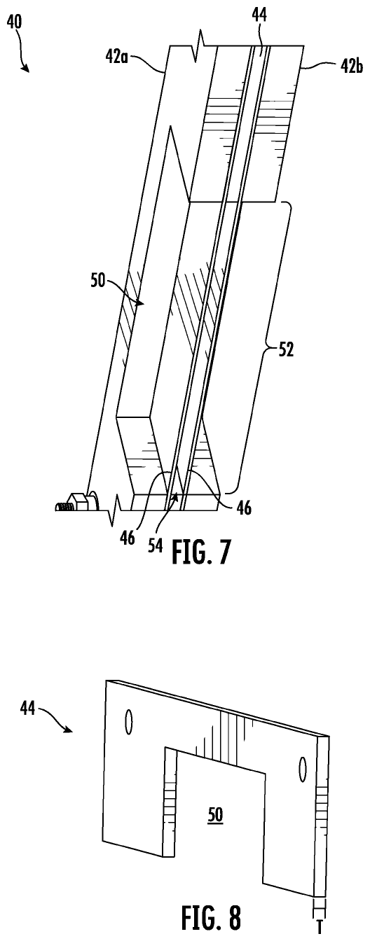

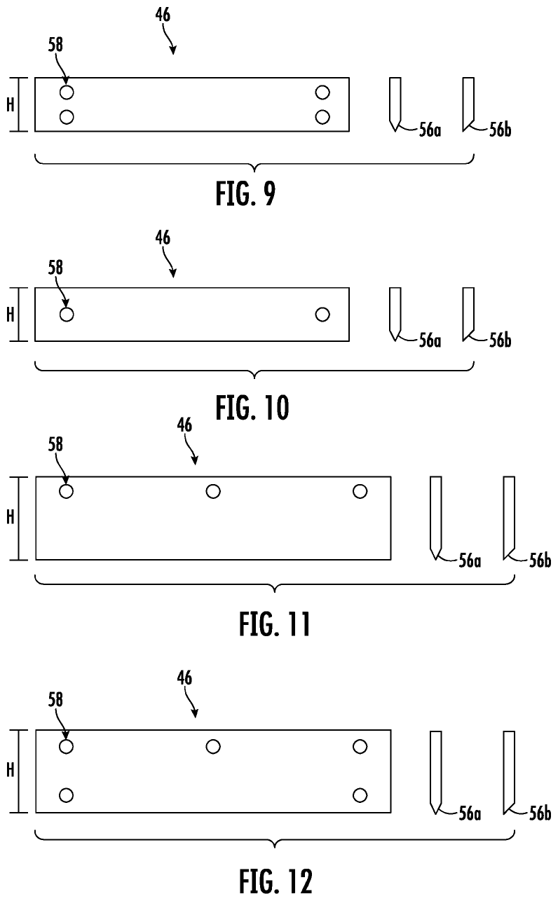

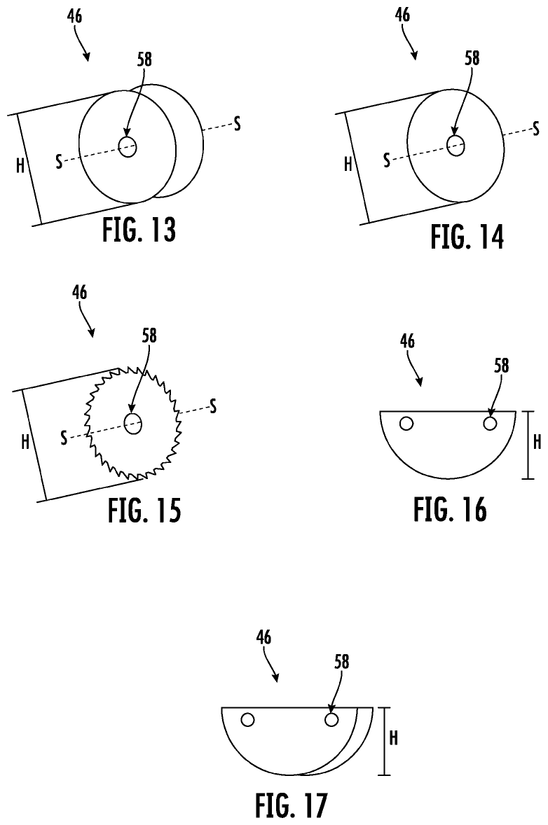

Referring to FIGS. 1, 2, 7, and 8, the cutting arm 40 cuts a piece of graft material from the piece of donor material 12. The cutting arm 40 includes a first arm member 42a, a second arm member 42b, a spacer 44, and a pair of blades 46. The cutting arm 40 is movably coupled to base 20 by the first arm member 42a and the second arm member 42b coupled to the legs 24 of the base 20 within the slots 26 by a pin 28. The pin 28 is disposed through the slots 26 and the first and second arm members 42a, 42b such that the cutting arm 40 may translate within the slots 26 or rotate about a pivot axis P-P defined by the pin 28 with respect to the base 20. The pin 28 may resist lateral translation of the cutting arm 40 such that the cutting arm 40 remains centered between the legs 24. The first arm member 42a, the second arm member 42b, and spacer 44 each define a cut-out 50 that align exposing the pair of blades 46 to form a cutting zone 52.

The cutting arm 40 is assembled in layers with the first arm member 42a and the second arm member 42b forming the outer most layers. The blades 46 and the spacer 44 are disposed between the first and second arm members 42a, 42b. The blades 46 are separated by the spacer 44. In order, layers of the cutting arm 40 are: one of the first arm member 42a or the second arm member 42b, a first blade of the pair of blades 46, the spacer 44, a second blade of the pair of blades 46, and the other of the first arm member 42a or the second arm member 42b. The pair of blades 46 may be parallel with each other. Within the cutting zone 52, a graft pocket 54 is defined between the pair of the blades 46 and the spacer 44. When cutting the donor material 12 to cut or carve a piece of graft material, the graft material is disposed between the pair of blades 46 within the graft pocket 54. The cut thickness of the cutting arm 40, and thus the resulting thickness of the graft material, is defined by the thickness of the spacer 44 and the resulting width of the graft pocket 54. The spacer 44 may have a thickness T in the range of 0.5 millimeters to 5 millimeters, e.g., 1.5 millimeters. In some embodiments, the cutting arm 40 includes a plurality of spacers 44 that are stackable with one another to adjust the width of the graft pocket 54, and thus the cut thickness of the cutting arm 40 and the graft material. For example, each spacer 44 may have a thickness of 0.5 millimeters and may include six spacers 44 to give an adjustable cut thickness in the range of 0.5 millimeters to 3 millimeters. In certain embodiments, each spacer 44 of the plurality of spacers 44 has a different thickness T.

In certain embodiments, the graft cutting device 10 includes a graft ejector (not shown). The graft ejector is configured to assist in the removal of the graft material from the graft pocket 54 after a clinician has made a cut. The graft ejector extracts or ejects the graft material from the graft pocket 45 without damaging or deforming the piece of graft material. The graft ejector may be disposed on the cutting arm 40 and may include a plunger and an ejector pin. The graft ejector may translate between a withdrawn state in which the ejector pin is removed from the graft pocket 54 and an inserted state in which the ejector pin is disposed within the graft pocket 54. The graft ejector may be biased toward the withdrawn state by a spring. To extract a piece of graft material from the graft pocket 54, the plunger may be depressed by a clinician to translate the graft ejector from the withdrawn state to the inserted state such that the ejector pin is inserted into the graft pocket 54 and contacts the graft material to urge the graft material from the graft pocket 54.

In embodiments, the graft cutting device 10 may include a cut adjustment mechanism. The cut adjustment mechanism may provide for adjustment of the cut thickness without the need to disassemble the cutting arm 40. The cut adjustment mechanism may have an adjustment screw configured to increase or decrease the distance between the pair of blades 46 and/or the first arm member 42a and the second arm member 42b to adjust the cut thickness. The adjustment screw of the cut adjustment mechanism may be indexed such that one complete turn increases or decreases the cut thickness a known amount. For example, one full clockwise turn of the adjustment screw may increase or decrease the cut thickness in 0.5 millimeter increments.

Referring now FIGS. 9-17, each blade of pair of blades 46 may have a variety profiles and bevels. For example, the blades 46 may have a rectangular profile and cross-section (FIGS. 9-12). In some embodiments, the blades 46 have a circular profile (FIGS. 13-15) or a semi-circular profile (FIGS. 16-17). The blades 46 may have a double beveled cutting edge 56a or a single beveled cutting edge 56b. In certain embodiments, the blades 46 have a serrated or saw-like cutting edge (FIG. 15). The blades 46 include an array of mounting holes 58 for securement to the cutting arm 40. The blades 46 have a height H to accommodate a piece of donor material 12 of various dimensions. For example, the blades 46 having a greater height H may allow the cutting arm 40 to cut entirely through a relatively large piece of donor material 12 in a single cut.

In some embodiments, where the blades 46 have circular profile and include a single mounting hole 58, the single mounting hole 58 may define a spin axis S-S. The circular blades 46 may spin about the spin axis S-S as the blades 46 cut through the donor material 12. Spinning of the blades 46 during cutting may reduce the chance of deforming the donor material 12 during cutting.

In particular embodiments, the cutting arm 40 may include only a single blade 46. The single blade 46 may be positioned between the first arm member 42a and the second arm member 42b, without a spacer 44. The spacer 44 may be included to perform an off-center cut. In certain embodiments a single blade 46 may be used to further trim or shape a piece of graft material.

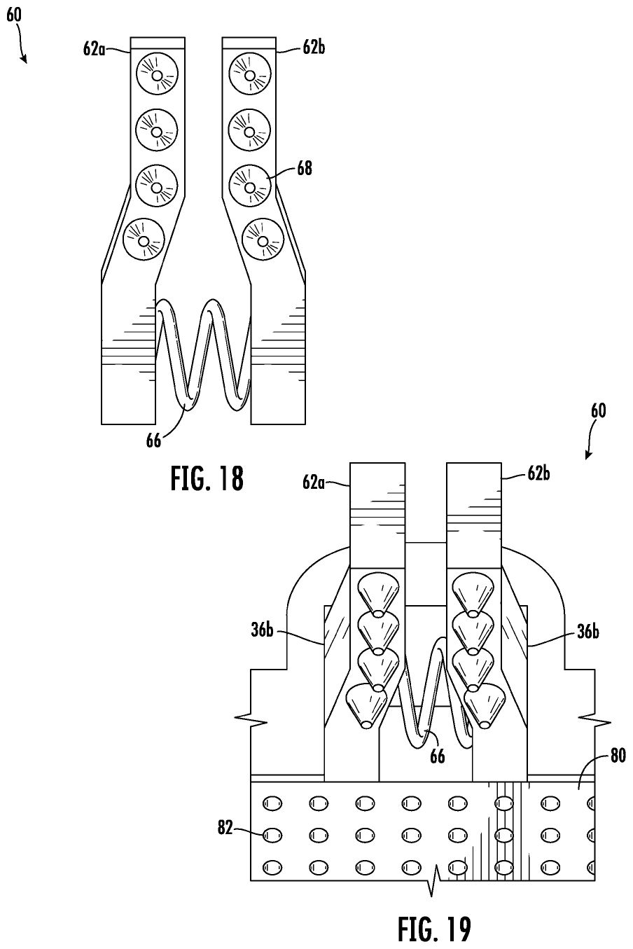

Referring to FIGS. 1, 2, 18, and 19, the fixators 60 engages the donor material 12 to fix the donor material 12 to the cutting board section 30 for cutting a graft. The fixators 60 fix the donor material 12 such that deformation of the donor material 12 as a result of fixation is minimized and the dimensions of the donor material 12 are retained while fixed to the cutting board section 30. In other words, the fixators 60 fix the donor material 12 so as to not overly deform the donor material 12. Deformation of the donor material 12 due to fixation may distort the dimensions of the resulting graft material and may require that the graft material be additionally trimmed to achieve the desired size and dimension or that an entirely new piece of graft material be cut.

The fixators 60 include a first holding member 62a, a second holding member 62b, and a spring 66. The spring 66 extends between the first holding member 62a and the second holding member 62b and urges the first holding member 62a and the second holding member 62b apart. The first and second holding member 62a, 62b each include a plurality of teeth 68. The teeth 68 project from the first and second holding member 62a, 62b and engage the donor material 12 to fix it to the cutting board section 30. The teeth 68 may have a conical profile. In some embodiments, the teeth 68 may have a pyramidal profile. In embodiments, the teeth 68 may have a frustoconical or frustropyramidal profile. In certain embodiments, the first and second holding members 62a, 62b may be formed as flat bumpers and omit the teeth 68. The first holding member 62a and the second holding member 62b may be made from a high friction material, e.g., a silicone-based material, to fix the donor material 12 in place for cutting while minimizing any clamping forces that may cause deformation of the donor material 12. The fixators 60 may be single use, disposable components of the graft cutting device 10. In some embodiments, the fixators 60 may be sterilizable so as to be reusable.

Referring to FIGS. 1, 2, and 19, the cutting bed 80 is inserted into the bed chamber 31 of the cutting board section 30 and directly supports the donor material 12 for cutting. A top surface of the cutting bed 80 may be coplanar with the top surface 38 of the cutting board section 30. In some embodiments, the top surface of the cutting bed 80 may be raised above the top surface 38 or may be recessed below the top surface 38. The cutting bed 80 may include nubs 82, similar to the teeth 68 of the fixators 60, to resist movement of the donor material 12 and aid in fixation. The nubs 82 may raise the donor material 12 to create clearance between the cutting bed 80 and the donor material 12 such that the blades 46 may cut entirely through the donor material 12 before contacting the cutting bed 80. The nubs 82 may have a conical profile or a hemi-spherical profile. In some embodiments, the nubs 82 may have a pyramidal profile. In embodiments, the teeth 68 may have a frustoconical or frustropyramidal profile. The cutting bed 80 may be made from a high friction material, e.g., a silicone-based material, to aid in fixing the donor material 12 in place for cutting. The cutting bed 80 may be a single use, disposable component of the graft cutting device 10. In some embodiments, the cutting bed 80 may be sterilizable so as to be reusable.

In embodiments, the graft cutting device 10 may form a graft cutting kit. The graft cutting kit may be aseptically sealed in anticipation of surgery in a single package pre-assembled for use by a clinician. In some embodiments, the graft cutting kit may include consumable components aseptically sealed and packaged separately from the reusable components, e.g., the base 20 of the graft cutting device 10. For example, the graft cutting kit may include a plurality of fixators 60, a cutting bed 80, a plurality of blades 46, and a plurality of spacers 44. The graft cutting kit may be assembled by a clinician with desired type of blades 46 and the desired number of spacers 44.

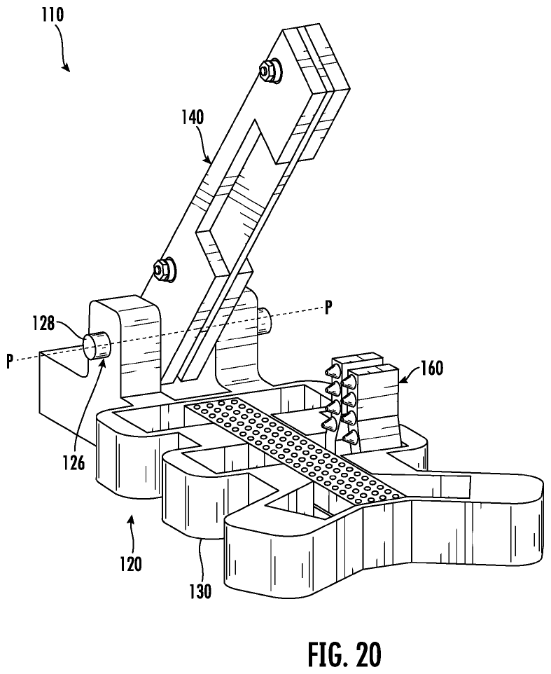

With reference to FIG. 20, another graft cutting device 110 is shown. The graft cutting device 110 is similar to the graft cutting device 10. As such, like elements will be labeled with a similar label including a preceding “1”. For the sake of brevity, only the differences between the graft cutting device 10 and the graft cutting device 110 will be described in detail.

The graft cutting device 110 includes a base 120 and a cutting arm 140. The cutting arm 140 pivotally couples to the base 120. The base 120 defines a through hole 126 to receive a pin 128 of the cutting arm 140 such that the cutting arm 140 may only rotate or pivot about the pivot axis P-P with respect to the base 120.

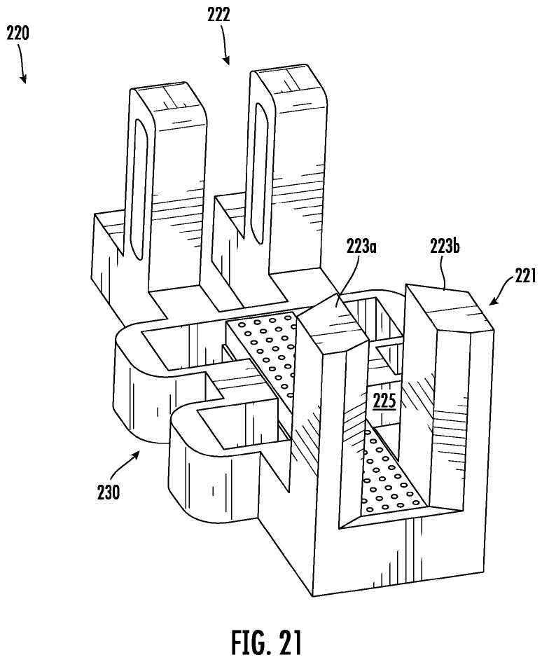

With reference to FIG. 21, another base 220 for a graft cutting device 210 in accordance with embodiments of the present disclosure is shown. The base 220 is similar to the base 20 of graft cutting device 10. As such, like elements will be labeled with a similar label including a preceding “2”. For the sake of brevity, only the differences between the base 20 and the base 220 will be described in detail.

The base 220 includes a cut guide 221 to assist a clinician in cutting graft material from a piece of donor material 12. The cut guide 221 includes a pair of guide rails 223a, 223b that are spaced apart to define a guide channel 225. When making a cut, a portion of the cutting arm 240 (not shown) passes between the pair of guide rails 223a, 223b within the guide channel 225. The cutting arm 240 may slidingly engage the guide rails 223a, 223b during a cut to assist the clinician in making a straight cut.

In some embodiments, the guide rails 223a, 223b include rollers (not shown) to assist the cutting motion and passage of the cutting arm 40 through the guide rails 223a, 223b. The rollers may be spring loaded and biased into the guide channel 225 such that the width of the guide channel 225 is variable and the rollers contact the cutting arm 40 at each cut thickness setting. For example, the rollers may extend from the guided rails 223a, 223b into the guide channel 225 a first distance when the cutting arm 240 is outside the guide channel 225. When the cutting arm 40 is set to cut a piece of graft material with a thickness of 0.5 millimeters, entry of the cutting arm 40 into the guide channel 225 may depress the rollers such that the rollers extend into the guide channel 225 a second distance that is less than the first distance. When the cutting arm 40 is set to cut a piece of graft material with a thickness of 2.5 millimeters, entry of the cutting arm 40 into the guide channel 225 may depress the rollers such that the rollers extend into the guide channel 225 a third distance that is less than the second distance.

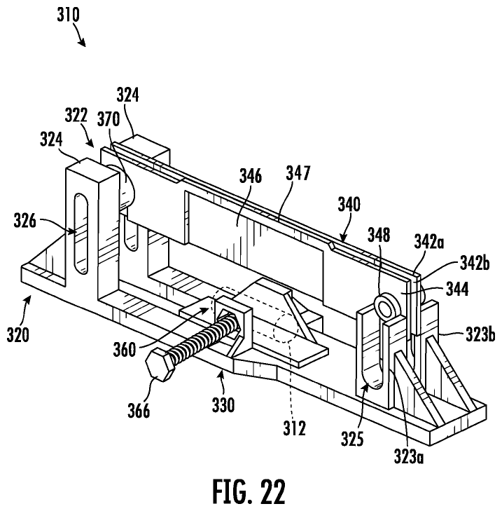

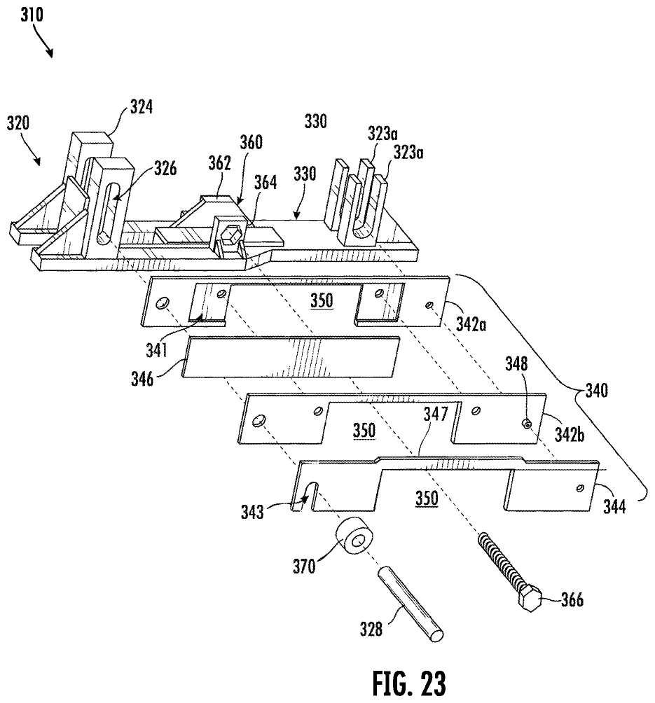

With reference to FIGS. 22-24, another graft cutting device 310 in accordance with embodiments of the present disclosure is shown. The graft cutting device 310 is similar to the graft cutting devices 10 and 110 and contains features similar to those of the base 220. As such, like elements will be labeled with a similar label including a preceding “3”. For the sake of brevity, only the differences between the graft cutting devices 10, 110, and the base 220 and the graft cutting device 310 will be described in detail.

The cutting arm 340 of the graft cutting device 310 includes a first arm member 342a, a second arm member 342b, a blade 346, a spacer 344, and a take-up washer 370. The first and second arm members 342a, 342b each include a knob 248 projecting outwardly therefrom. The knob 348 is received within the guide channels 325 of the cut guides 323a, 323b of the base 320. The first and second arm members 342a, 342b hold the blade 346 therebetween. The first or the second arm member 342a, 342b may define a blade recess 341 to receive and hold the blade 346 therein.

The cutting arm 340 includes a spacer 344 to adjust the cut thickness of the cutting arm 340 and the resulting thickness of the graft material. The spacer 344 defines notch 343 and a knob hole 345 and includes a tab 347. The spacer 344 is placed adjacent to the first arm member 342a or the second arm member 342b, as opposed the spacer 44 of the graft cutting device 10 disposed between the first and second arm members 42a, 42b. The spacer 344 defines the notch 343 to receive a portion of the pin 328 and defines the knob hole 345 to receive the knob 348 of the first arm member 342a or the second arm member 342b therethrough. The knob hole 345 may be sized and dimensioned to form a friction fit with the knob 348 to secure the spacer 344 to the first or second arm member 342a, 342b. The spacer 344 may be secured to the first arm member 342a or the second arm member 342b without decoupling the cutting arm 340 from the base 320. The tab 347 may provide a purchase point to assist in removal and securement of the spacer 344. The cut thickness may be adjusted by reconfiguring the cutting arm 340 such that the spacer 344 is secured to the other of the first arm member 342a or the second arm member 342b. For example, when the spacer 344 is secured to the first arm member 342a the cutting arm 340 may have a thicker cut thickness than when the spacer 344 is secured to the second arm member 342b.

The take-up washer 370 is disposed on the pin 328 and takes up excess space between the spacer 344 or the first or second arm members 342a, 342b and the legs 324 of the base 320. The take-up washer 370 maintains alignment of the cutting arm 340 relative to the donor material 12 so that graft material cut from the piece of donor material 312 has a uniform thickness. The take-up washer 370 may be made in multiple heights to take up more or less excess space and to accommodate spacers 344 of various thickness. In some embodiments, the take-up washer 370 has a variable height such that a single take-up washer 370 may be compatible with multiple spacers 344 of various thickness. In such embodiments, the take-up washer 370 may include a spring to urge the cutting arm 340 into proper alignment.

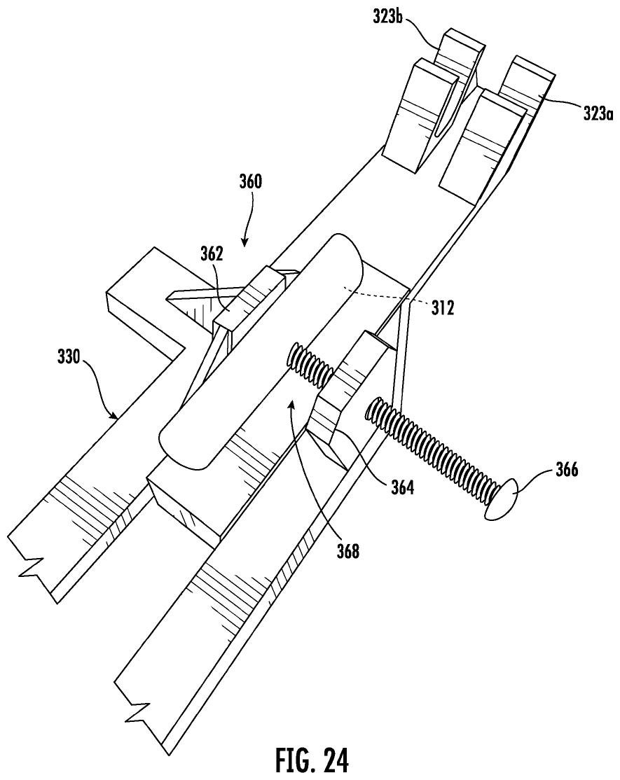

With particular reference to FIG. 24, the graft cutting device 310 includes a clamp 360 to fix the piece of donor material 312 for cutting a piece of graft material without deforming the donor material 312. The clamp 60 includes a stopper wall 362, a clamp wall 364, and a clamp screw 366. The stopper wall 362 and the clamp wall 364 define a clamp chamber 368 to receive the donor material 312. The clamp screw 366 is threadably coupled to the clamp wall 364 such that rotation of the clamp screw 366 disposes or withdraws a portion of the clamp screw 366 into or out of the clamp chamber 368. The clamp screw 366 engages the piece of donor material 312 to clamp the donor material 312 against the stopper wall 362 and fixate the donor material 312 for cutting. In some embodiments, the clamp screw 366 may include a clamp boot (not shown) to distribute the clamping forces across a greater area of the donor material 312. The clamp boot may further reduce the chance of deforming the donor material 312.

The cut thickness of the cutting arm 340 is the distance between the stopper wall 362 and the blade 346. As described above, the thickness of the spacer 344 and its position in the cutting arm 324, secured to the first arm member 342a or the second arm member 342b, defines the cut thickness. Positioning the spacer 344 closer to the stopper wall 362 may shift the blade 346 further from the stopper wall 362 and, thus, increase the cut thickness. Positioning the spacer 344 further from the stopper wall 362 may shift the blade 346 closer to the stopper wall 362 and, thus, decrease cut thickness. The cutting arm 340 may be disposed between the legs 324 and the cut guides 323a, 323b and alighted relative to the stopper wall 362 such that the blade 346 is parallel with the stopper wall 362.

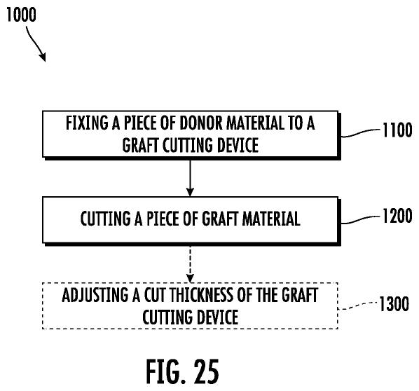

With reference to FIG. 25, a method 1000 of using a graft cutting device in accordance with embodiments of the present disclosure is described with reference to the graft cutting device 10 of FIGS. 1-19.

A piece of donor material 12 is secured to the cutting board section 30 of the graft cutting device 10 (Step 1100). The donor material 12 is placed on the cutting bed 80. The fixators 60 are positioned within the receptacles 32 such that the teeth 68 engage the donor material 12 to fix it in place without deforming the donor material 12. The fixators 60 are secured in place within a respective receptacle 32 of the plurality of receptacles 32 by the spring 66 urging the first holding member 62a and the second holding member 62b against the side walls 36b of the receptacle 32. The fixators 60 may be positioned within the receptacle 32 to accommodate various sized pieces of donor material 12. The fixators 60 may be positioned at any point along the length L of the receptacle 32. To adjust the position of the fixators 60 within the receptacle 32, the first holding member 62a and the second holding member 62b may be pressed towards each other, compressing the spring 66, to disengage the first and second holding members 62a, 62b from the side walls 36b and the fixator 60 moved along the length L toward or away from the cutting platform 34, and thus the donor material 12.

The secured piece of donor material 12 is cut to obtain a piece of graft material by moving the cutting arm 40 from the open position to the closed position (Step 1200). The cutting arm 40 may move between the open position and the closed position and cut graft material from the piece of donor material 12 by chopping, slicing, or a combination of chopping and slicing. Chopping includes cutting graft material by passing the blades 46 straight through the piece of donor material 12 such that the depth of the cut is uniform along the length of the blades 46 throughout the duration of the cut. During chopping the cutting arm 40 translates with respect to the base 20 with only minimal rotational motion, keeping the edges 56 of the blades 46 substantially parallel to the top surface 38 of the base 20. Slicing includes cutting graft material by passing the blades 46 through the piece of donor material 12 at an angle such that the depth of the cut varies along the length of the blades 46 throughout the duration of the cut. During slicing the cutting arm 40 rotates or pivots about the pin 28 with respect to the base 20 with minimal translation such that the edges 56 of the blades 46 remain askew relative to the top surface 38 of the base 20 until the cutting arm 40 reaches the closed position. Once a piece of graft material has been cut, the piece of graft material may be removed by returning the cutting arm 40 to the open position, leaving the piece of graft material on cutting board section 30. In embodiments, the piece of graft material may be removed from the graft pocket 54 the aid of a tool, e.g., a surgical tool found in an operating room. In certain embodiments, the piece of graft material may be removed from the graft pocket 54 by disassembling or partially disassembling the cutting arm 40.

In some embodiments, the cut thickness of cutting arm 40 may be adjusted to cut another piece of graft material of a different dimension (Step 1300). The pin 28 may be removed from the slots 26 to decouple the cutting arm 40 from the base 20. The cutting arm 40 may be completely or partially disassembled to add or remove a spacer 44. The first arm member 42a or the second arm member 42b and one of the blades 46 may be removed. Another spacer 44 may be added to space the blades 46 further apart and the cutting arm 40 reassembled, thus, increasing the cut thickness, or a spacer 44 may be removed to space the blades 46 closer together and decrease the cut thickness.

Although the method steps are described in a specific order, it should be understood that other steps may be performed in between described steps, described steps may be adjusted so that they occur at slightly different times, or the described steps may occur in any order unless otherwise specified.

While several embodiments of the disclosure have been shown in the drawings, it is not intended that the disclosure be limited thereto, as it is intended that the disclosure be as broad in scope as the art will allow and that the specification be read likewise. Any combination of the above embodiments is also envisioned and is within the scope of the appended claims. Therefore, the above description should not be construed as limiting, but merely as exemplifications of particular embodiments. Those skilled in the art will envision other modifications within the scope of the claims appended hereto.

What is claimed is:

1. A graft cutting device comprising:
a base including a cutting board section and a pair of legs, the cutting board section defining a plurality of receptacles and the pair of legs each defining a slot;
a cutting arm disposed between the pair of legs and moveably coupled to the base within the slots such that the cutting arm rotates or translates with respect to the base between an open position in which the cutting arm is disengaged from the cutting board section and a closed position in which the cutting arm is engaged with the cutting board section, the cutting arm comprising:
a first arm member;
a second arm member;
a first blade;
a second blade, the first blade and the second blade disposed between the first arm member and the second arm member; and
a spacer disposed between the first blade and the second blade, the first blade and the second blade spaced apart by the spacer; and
a plurality of fixators, each fixator received within a respective receptacle of the plurality of receptacles, the plurality of fixators configured to fix a piece of donor material to the cutting board section such that the dimensions of the piece of donor material are retained while fixed to the cutting board section.

2. The graft cutting device according to claim 1, wherein each fixator of the plurality of fixators comprises a first holding member, a second holding member, and a spring extending between the first holding member and the second holding member.

3. The graft cutting device according to claim 2, wherein the spring urges the first holding member and the second holding member apart such that when the fixator is received within a respective receptacle of the cutting board section the fixator is adjustably secured to the base within the receptacle by the first holding member and the second holding member urged against a pair of side walls of the receptacle by the spring.

4-5. (canceled)

6. The graft cutting device according to claim 1, comprising a cutting bed disposed within a bed chamber defined between a top surface of the cutting board section and a bed platform of the cutting board section.

7. The graft cutting device according to claim 6, wherein a top surface of the cutting bed is coplanar with the top surface of the cutting board section.

8. The graft cutting device according to claim 6, wherein a top surface of the cutting bed is recessed below the top surface of the cutting board section or raised above the top surface of the cutting board section.

9. The graft cutting device according to claim 1, wherein the first arm member, the second arm member, and the spacer each define a cut-out to expose a portion of the first blade and the second blade to form a cutting zone of the cutting arm.

10. The graft cutting device according to claim 9, wherein the first blade, the second blade, and the spacer define a graft pocket therebetween, the graft pocket having a width that defines a thickness of a piece of graft material cut from the piece of donor material, the width of the graft pocket defined by a thickness of the spacer.

11-15. (canceled)

16. A graft cutting device comprising:
a base having a cutting board section;
a cutting arm movably coupled to the base, the cutting arm configured to cut a piece of graft material from a piece of donor material; and
a plurality of fixators adjustably secured to the base, the fixators configured to fix the piece of donor material to the cutting board section such that the piece of donor material is undeformed by the fixators while fixed to the cutting board section.

17. The graft cutting device according to claim 16, wherein the cutting arm comprises a spacer configured to define a thickness of the piece of graft material cut from the piece of donor material.

18. The graft cutting device according to claim 16, wherein the base defines a plurality of receptacles to receive a fixator of the plurality of fixators therein, each receptacle having a length, the fixator secured to the base within the receptacle such that the position of the fixator within the receptacle is adjustable along the length of the receptacle.

19. The graft cutting device according to claim 16, wherein each fixator comprises a spring configured to urge a portion of the fixator against a pair of side walls of a receptacle such that the fixator is adjustably secured to the base.

20. (canceled)

21. The graft cutting device according to claim 16, wherein the base comprises a pair of legs extending outwardly therefrom, each leg of the pair of legs defining a slot, the cutting arm movably coupled to the pair of legs within each slot such that the cutting arm is capable of rotating or translating with respect to the base.

22. The graft cutting device according to claim 16, wherein the base includes a plurality of clamp points configured to allow for securement of the base to a work surface.

23. The graft cutting device according to claim 16, wherein the base comprises a cut guide configured to align a blade of the cutting arm relative to the piece of donor material such that the piece of graft material cut therefrom has a uninform thickness.

24. A fixator for a graft cutting device comprising:
a first holding member;
a second holding member; and
a spring extending between the first holding member and the second holding member, the spring urging the first holding member and the second holding member away from each other, the fixator configured to fix a piece of donor material to the graft cutting device such that the piece of donor material is undeformed by the fixator.

25. The fixator according to claim 24, wherein the first holding member and the second holding member are made from a high friction material.

26. The fixator according to claim 25, wherein the high friction material is a silicone-based material.

27. The fixator according to claim 24, wherein the first holding member and the second holding member each include a plurality of teeth projecting therefrom.

28. The fixator according to claim 27, wherein each tooth of the plurality of teeth of the first holding member and the second holding member has a conical profile.

29-38. (canceled)
